(19) United States
(12) Patent Application Publication
(10) Pub. No.: US 2014/0110289 A1
(43) Pub. Date: Apr. 24, 2014

(54) SYRINGE FOR INJECTING A SURGICAL GAS

(71) Applicant: Fluoron GmbH, Ulm (DE)

(72) Inventor: Volker Geuder, Ulm (DE)

(21) Appl. No.: 14/060,317

(22) Filed: Oct. 22, 2013

(30) Foreign Application Priority Data
Oct. 22, 2012 (DE) 20 2012 104055.7
Nov. 29, 2012 (DE) 20 2012 104643.1

(51) Int. Cl. A61F 9/007; B65B 3/00

(52) U.S. Cl. CPC A61F 9/007; B65B 3/003
USPC 206/364; 604/24; 141/2

(57) ABSTRACT

The invention relates to a ready-to-use syringe containing a mixture of air and a heavy gas in a predetermined mixing ratio. The syringe is for use as a gas tamponade system for medical applications. The tamponade is used to fill a natural cavity resulting e.g. from the shrinkage of a vitreous humour due to ageing, or an artificial cavity resulting e.g. from a vitrectomy.

### FIELD OF THE INVENTION

The invention relates to a ready-to-use gas tamponade system for medical applications. The tamponade is used to fill a natural cavity resulting e.g. from the shrinkage of a vitreous humour due to ageing, or an artificial cavity resulting e.g. from a vitrectomy. The invention further relates to a system consisting of a bag and a syringe packed inside.

### BACKGROUND OF THE INVENTION

Increasing human life expectancy is giving rise to an increase in age-related diseases such as retinal changes or retinal detachments, glaucoma, cataract, and age-related macular degeneration and diabetic retinopathy. Treatment of these and other eye diseases usually requires a vitrectomy (removal of the vitreous humour). The resulting cavity has to be refilled to prevent the vitreous chamber from collapsing. This is done using “heavy gases” such as SF₆, C₂F₆ or C₃F₈. The term “heavy gases” is generally used to refer to gases which have a markedly higher density than normal atmospheric air.

In most cases the therapeutic effect is due not to the gas itself, but rather to the gas-liquid interface. This surface tension prevents gas from entering the subretinal space through a hole in the retina; in addition, the hole in the retina is expanded, preventing further liquid from entering the subretinal space.

After injection of the heavy gas, O₂ and CO₂ start to diffuse out of the blood and into the eye, increasing the volume of the gas bubble. A diffusion equilibrium is reached after a few hours for O₂ and CO₂, but only after a few days for N₂. The heavy gases are absorbed through the retina via the choroid membrane and, depending on the type, leave the eye within 1 to 2 or 4 weeks. In most cases to date, the gases used in surgery, such as SF₆, C₃F₈ or C₂F₆, have been transferred directly during the operation from a steel cylinder into the medium to be used. U.S. Pat. No. 6,866,142 B2 or 6,073,759 A describes a system consisting of an already gas-filled disposable syringe which, for better gas tightness, is stored in a container filled with the same gas as the disposable syringe.

U.S. Pat. No. 6,599,280 B1 and published specification FR 2 020 245 A both describe a surgical kit with a separate gas reservoir, a syringe and other components usable in combination with the syringe, which are housed in a pack.

All the systems listed above have several disadvantages.
In all the systems, SF₆, C₂F₆ and C₃F₈ are released into the environment, sometimes in considerable amounts, even though these gases are among the most potent greenhouse gases known; thus, for example, 1 kg of SF₆ has the same effects as 22.2 t of CO₂ [EC Regulation No. 842/2006].
Gas tamponades are not allowed to be filled from the steel cylinder in the OR because steel cylinders have to be stored in a special steel cabinet for pressurized containers.
The single doses available on the market have to be prepared in several steps before the surgeon can use them.

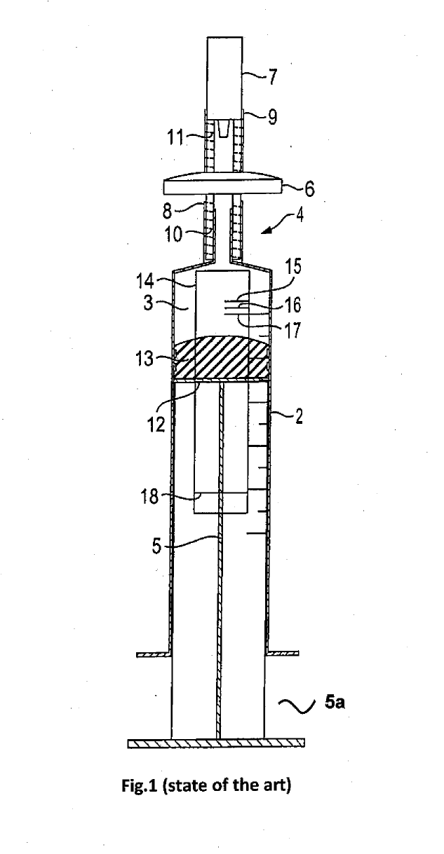

To solve these problems, European patent specification EP 2 319 566 B1 discloses the following syringe for injecting a surgical gas, shown in FIG. 1:

This is a syringe (1), in the form of a ready-to-use system,
with a syringe body (2) having
   an inner chamber (3) and
   a tip (4) connected to the inner chamber (3),
with a plunger rod (5) guided in a longitudinally displaceable manner in the inner chamber (3), and
with a sealing stopper (7),
characterized in that the syringe is in the form of a ready-to-use system,
the inner chamber (3) taking the form of a gas container and being filled with a heavy gas,
a syringe attachment filter (6) being fixed to the tip (4), and
the sealing stopper (7) being fixed to the syringe attachment filter (6), which seals the inner chamber (3) gas-tight.

Such a syringe is used as follows. First the inner chamber of the syringe body is filled with a heavy gas, such as SF₆, C₃F₈ or C₂F₆, in an amount greater than the desired (necessary) amount of heavy gas that is required to establish a desired mixing ratio of the heavy gas with air. The filled syringe is delivered in this form to the user (e.g. eye surgeon).

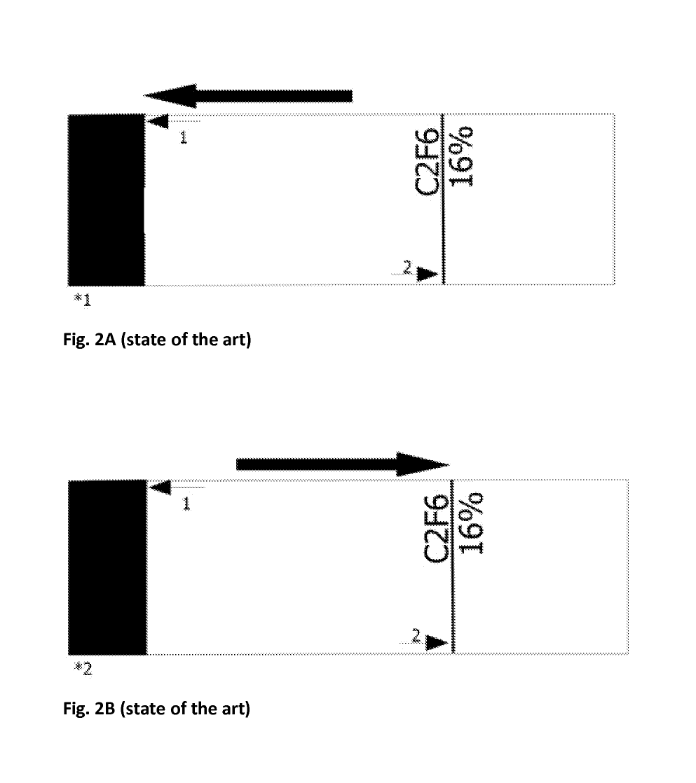

The necessary gas/air ratio must then still be established (by the user) before the syringe is used. This is done by pushing the syringe plunger forward (by means of the plunger rod 5) to a specified mark. Excess heavy gas is thereby expelled from the inner chamber (3). In the syringe of FIG. 1 a label (14) with mixing ratio marks (15, 16 or 17) for different heavy gases, such as SF₆, C₃F₈ or C₂F₆, is affixed to the syringe body (2) for this purpose. In FIG. 2A this mark is characterized by the reference (*1). The plunger is then pulled back to the second mark (reference (*2) in FIG. 2B). This is how the desired mixing ratio is established (e.g. 16% C₂F₆/84% air, as shown in FIG. 2). After the desired gas/air mixture has been established, the syringe attachment filter (6) is removed and a desired cannula, fitted with the protective cap, is attached. After the vitrectomy has been performed, the mixture of heavy gas and air is used as the medium for the long-term tamponade. The heavy gas has reached its full expansion about 72 hours after the vitrectomy has been performed and is not removed, but replaced with endogenous liquids and exhaled over a period of up to 30 days.

It would be desirable, however, to provide a ready-to-use syringe (i.e. a syringe in the form of a ready-to-use system) which allowed even easier handling for surgical use. In the case of prolonged storage of the syringe prior to use in the eye operation, it is also possible for air to diffuse into the inner chamber of the syringe body (plastic materials are known to be permeable to gases like oxygen or nitrogen), thereby altering the amount of heavy gas in the inner chamber. This could then lead to deviations from the desired mixing ratio of heavy gas to air.

The object of the present invention is therefore to provide a syringe of the type indicated in the pre-characterizing clause of Claim 1 which is capable of mitigating the disadvantages of the state of the art illustrated above and, in particular, is simple to operate.

### DETAILED DESCRIPTION OF THE INVENTION

This object is achieved by the syringe according to the invention and a bag with a syringe packed inside which has the features of the independent claims. The respective subordinate claims contain advantageous additional forms of the invention.

In principle, various embodiments of the syringe according to the invention are covered by the present invention. Thus, preferably, the inner chamber of the syringe can be filled with a mixture of air and heavy gas in a predetermined mixing ratio, or alternatively just with heavy gas. Both embodiments are aspects of the same invention and solve the aforementioned technical problem insofar as they provide a syringe according to the invention which is ready to use and simple to operate and allows the preparation of a desired mixing ratio of heavy gas and air.

In one preferred embodiment, such a syringe is a syringe (1) in the form of a ready-to-use system
with a syringe body (2) having
   an inner chamber (3) and
   a tip (4) connected to the inner chamber (3),
with a plunger (5a), guided in a longitudinally displaceable manner in the inner chamber (3), preferably with a plunger rod (5), and
with a sealing stopper (7),
the inner chamber (3) taking the form of a gas container and being filled with a mixture of air and a heavy gas in a predetermined mixing ratio, the plunger rod (5) or plunger (5a), respectively, being positioned in such a way that the mixture of air and heavy gas present in the predetermined mixing ratio is capable of immediate administration,
a syringe attachment filter (6) being fixed to the tip (4), and
the sealing stopper (7) being fixed to the syringe attachment filter (6), which seals the inner chamber (3) gas-tight,
the syringe body not carrying a mark (18) for the maximum plunger stroke and/or one or more mixing ratio marks (15, 16 or 17) for different heavy gases.

The expression “taking the form of a gas container” when used herein means “designed as a gas container” and/or “used as a gas container”, and implies that the inner chamber (3) preferably has characteristics which render it suitable for containing or holding back gas, e.g., that it can be closed in a gas-tight manner.

The expression “gas” in general comprises any substance in gaseous form, and in particular heavy gas and/or air as defined herein.

The sealing stopper is preferably fixed to the syringe attachment filter (6) and seals the inner chamber gas-tight, meaning that the sealing stopper (7) allows essentially no gas to exit and/or enter the inner chamber (3). However, this also implies that, when in a preferred embodiment of the invention, the ready-to-use-system comprises a syringe attachment filter, and the sealing stopper is affixed to the syringe attachment filter, “sealing the inner chamber gas-tight” also includes that gas may diffuse back and forth through the syringe attachment filter, but is prevented from entering or leaving the system by the sealing stopper.

In one alternative embodiment, such a syringe is a syringe (1) in the form of a ready-to-use system
with a syringe body (2) having
   an inner chamber (3) and
   a tip (4) connected to the inner chamber (3),
with a plunger (5a), guided in a longitudinally displaceable manner in the inner chamber (3), preferably with a plunger rod (5),
with a sealing stopper (7), and
with a mark (18) affixed to the syringe body (2) for the maximum plunger stroke,
the inner chamber (3) taking the form of a gas container and being filled with a predetermined amount of a heavy gas, the amount of gas producing a desired mixing ratio of the heavy gas with the aspirated air when the plunger rod (5) or plunger (5a), respectively, is directly pulled back to the mark (18) for the maximum plunger stroke, without the plunger rod (5) having been moved, prior to the aspiration of air, to a position in the inner chamber (3) where an excess amount of the heavy gas is expelled from the inner chamber (3) in order to establish the desired mixing ratio between the heavy gas and the air,
a syringe attachment filter 6 being fixed to the tip (4), and
the sealing stopper (7) being fixed to the syringe attachment filter (6), which seals the inner chamber (3) gas-tight.

As an alternative, the syringe attachment filter (6) can also be omitted, the sealing stopper (7) being fixed to the syringe in such a way that it seals the inner chamber (3) gas-tight. This alternative is applicable to all the embodiments described herein, e.g., embodiments pertaining to the syringe, bag or methods described herein.

“Seal gas-tight” means “seal in a manner such that essentially no gas is allowed to exit and/or enter”. Those skilled in the art are aware of the fact that it is technically impossible to produce a completely gas-tight seal that totally prevents the exchange of individual gas molecules over an unlimited period of time. The expression therefore covers a small loss of gas from the inner chamber.

A bag is a bag (20) with a syringe (1) packed inside, the latter being a syringe in the form of a ready-to-use system
with a syringe body (2) having
   an inner chamber (3) and
   a tip (4) connected to the inner chamber (3),
with a plunger (5a), guided in a longitudinally displaceable manner in the inner chamber (3), preferably with a plunger rod (5), and
with a sealing stopper (7),
a syringe attachment filter (6) being fixed to the tip (4),
the sealing stopper (7) being fixed to the syringe attachment filter (6), which seals the inner chamber (3) gas-tight, and
the inner chamber (3) taking the form of a gas container and being filled with a mixture of air and a heavy gas in a predetermined mixing ratio, the plunger rod (5) or plunger (5a), respectively, being positioned in such a way that the mixture of air and heavy gas present in the predetermined mixing ratio is capable of immediate administration.

Other details, features and advantages of the invention are evident from the following detailed description of the invention and individual Examples and with the aid of the drawings, in which:

FIG. 1 shows a ready-to-use syringe (syringe in the form of a ready-to-use system) known from European patent specification EP 2 319 566 B1.

FIGS. 2A and 2B illustrate, by way of a syringe filled with C₂F₆, the method of establishing the desired mixing ratio between the heavy gas and air in the syringe of FIG. 1. FIG. 2A shows the first step, in which excess heavy gas is expelled from the inner chamber of the syringe body by pushing the syringe plunger up to the mark *1, and the amount of heavy gas is reduced to the amount that is desired to establish the desired mixing ratio of heavy gas to air. FIG. 2B shows the second step, in which air is aspirated by moving the plunger back to the mark *2 in order to achieve the desired mixing ratio of heavy gas to air.

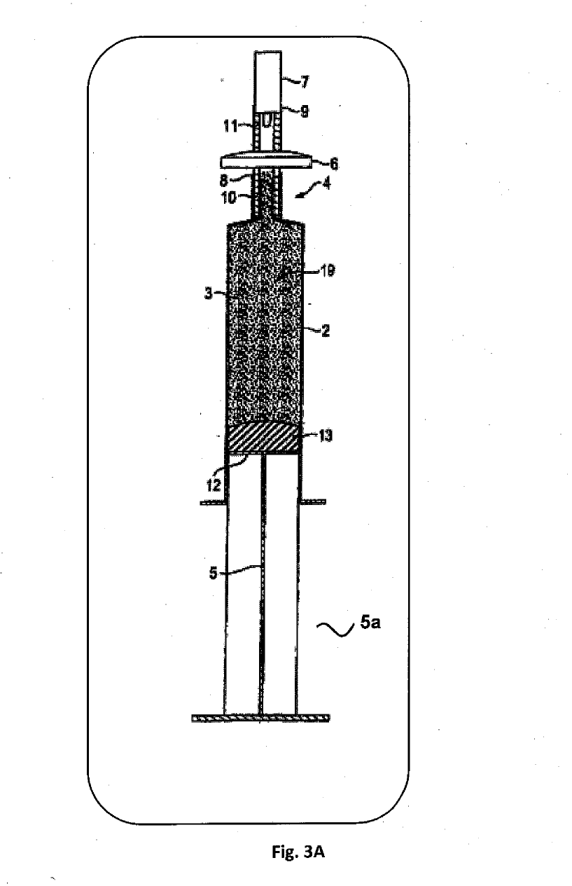

FIGS. 3A and 3B show embodiments of a syringe according to the invention. FIG. 3A illustrates a preferred embodiment of a syringe according to the invention and of a bag according to the invention with a syringe housed inside, the inner chamber of the syringe containing a mixture of heavy gas and air. FIG. 3B shows an alternative embodiment of a syringe according to the invention.

One preferred embodiment of the present invention is based on filling the inner chamber 3 of the syringe, in the form of a gas container, at the outset with a heavy gas and air in a predetermined mixing ratio so that the syringe containing the predetermined mixing ratio of the mixture of air and heavy gas is capable of immediate use. In other words, the invention makes it possible, by establishing the desired mixing ratio of heavy gas to air, to omit both the steps shown in FIGS. 2A and 2B that have to be performed by the user.

This preferred embodiment of the syringe according to the invention is substantially simpler to operate than syringes known from the state of the art, since the desired mixing ratio of heavy gas to air is established at the outset and no longer has to be established by the user. This in turn has the advantage that it is possible preferably to omit a mark (18) for the maximum plunger stroke and/or mixing ratio marks (15, 16 or 17) for different heavy gases, such as SF₆, C₃F₈ or C₂F₆, on the syringe body.

According to one alternative embodiment, the inner chamber 3 of the syringe, in the form of a gas container, is filled at the outset with a predetermined amount of a heavy gas, the amount of heavy gas immediately giving a desired mixing ratio of the heavy gas with the aspirated air when the plunger rod 5 or plunger 5a, respectively, is directly pulled back to the mark 18 affixed to the syringe body for the maximum plunger stroke. This embodiment makes it possible, by establishing the desired mixing ratio of heavy gas to air, to omit the first step shown in FIGS. 2A and 2B. This alternative embodiment of the syringe according to the invention accordingly makes it possible to establish the desired mixing ratio without the plunger rod 5 having been moved, prior to the aspiration of air, to a position in the inner chamber 3 where an excess amount of the heavy gas is expelled from the inner chamber 3 in order to establish the desired mixing ratio between the heavy gas and the air. Once again, this alternative embodiment is substantially simpler to operate than syringes known from the state of the art, since the desired mixing ratio of heavy gas to air can be established by pulling out the plunger 5a once and aspirating air.

In conformance with the above disclosure, all the embodiments of the syringe according to the invention accordingly have the advantage that it is possible preferably to omit mixing ratio marks (15, 16 or 17) for different heavy gases, such as SF₆, C₃F₈ or C₂F₆, on the syringe body. The syringe according to the invention therefore also does without a label 14 to which mixing ratio marks (15, 16 or 17) for different heavy gases are affixed. This not only simplifies the design and production of the syringe, but also has the advantage that the user cannot inadvertently establish an incorrect mixing ratio by accidentally moving the plunger 5a, when expelling the excess heavy gas, to a mark intended for a gas other than the one being used. This potential source of error in use is thus excluded by the syringe according to the invention. The various embodiments accordingly have the advantage that they are simpler to operate than the syringes known from the state of the art and that an excess amount of heavy gas does not have to be used (i.e. actually wasted) in order to fill them.

Two different embodiments of the syringe according to the invention are illustrated in FIGS. 3A and 3B. This shows embodiments of a syringe 1 according to the invention which have a cylindrical syringe body 2 comprising an inner chamber 3. The inner chamber 3 is connected to a tip 4, and a plunger rod 5 is guided in a longitudinally displaceable manner in the inner chamber 3.

As also illustrated in FIGS. 3A and 3B, a syringe attachment filter 6 is fixed to the tip 4. The entire system is further sealed gas-tight with a sealing stopper 7, which in turn can be fixed to the outer end of the syringe attachment filter 6.

In one particularly preferred embodiment, the syringe attachment filter 6 has a first fixing section 8 for attachment to the tip 4 and a second fixing section 9 for securing the sealing stopper 7. Preferably, the syringe attachment filter 6 and the sealing stopper 7 can be fixed by means of screw connectors 10 and 11 respectively. In one particularly preferred embodiment, the screw connectors 10 and 11 each take the form of Luer lock connectors.

As also illustrated in FIGS. 3A and 3B, at its end region 12 located in the inner chamber 3, the plunger rod 5 of the plunger 5a has a rubber stopper 13, which is preferably siliconized.

In the preferred embodiment of the syringe according to the invention shown in FIG. 3A, the plunger rod 5 or plunger 5a, respectively, is positioned in such a way that the mixture 19 of air and heavy gas present in the predetermined mixing ratio is capable of immediate administration. “Capable of immediate administration” means that the mixture of air and heavy gas is already present in the desired mixing ratio in the inner chamber 3 of the syringe body 2 and can be administered directly by the user, e.g. an ophthalmologist, for the therapeutic purpose of his choice without any further steps (such as the aspiration of air and expulsion of the excess amount of air to establish the desired mixing ratio of heavy gas to air). FIG. 3A also shows a bag 20 in which the syringe 1 has been housed (packed). The bag 20 is conventionally sealed air-tight and is also conventionally sterilized together with the syringe contained therein for the use of the syringe.

Apart from the conventional gaseous mixture of the earth's atmosphere, consisting mainly of the two gases nitrogen (around 78.08 vol %) and oxygen (around 20.95 vol %) and traces of argon, carbon dioxide, water vapour and other gases, the term “air” also includes synthetic mixtures of nitrogen and oxygen with other mixing ratios, e.g. 95 vol % of nitrogen and 5 vol % of oxygen, 80 vol % of nitrogen and 20 vol % of oxygen or 75 vol % of nitrogen and 25 vol % of oxygen. The term “air” also includes pure nitrogen in this context.

In contrast to the syringe shown in FIG. 1, no mark 18 is provided on the syringe body 2 for the maximum plunger stroke, nor are there any mixing ratio marks 15, 16 or 17 for different heavy gases, which, in the case of the syringe according to FIG. 1, can be affixed directly to the syringe body 2 or a label 14.

FIG. 3B shows an alternative embodiment of the syringe according to the invention in which a mark 18 is provided for the maximum plunger stroke. This mark 18 can be affixed either directly to the syringe body 2 or “indirectly” by way of a label (not shown in FIG. 3). Here again there are no mixing ratio marks 15, 16 or 17 for different heavy gases, which, in the case of the syringe according to FIG. 1, can be affixed directly to the syringe body 2 or a label 14.

As already explained at the outset, the syringe 1 according to the invention can take the form of a disposable syringe.

In all the embodiments, the heavy gas used in the syringe can be any gas suitable for eye operations. In preferred embodiments, the heavy gas is SF₆, C₃F₈ or C₂F₆. In the latter case, in embodiments such as those shown in FIG. 3A, the predetermined amount of air and heavy gas in the inner chamber 3 is preferably proportioned to give the following mixing ratio:
for SF₆: 20% gas/80% air,
for C₂F₆: 16% gas/84% air, and
for C₃F₈: 12% gas/88% air.

Alternatively, in embodiments such as those shown in FIG. 3B, the predetermined amount of heavy gas in the inner chamber 3 is preferably proportioned to give the aforementioned mixing ratios of the heavy gas with the aspirated air after the plunger rod has been pulled back.

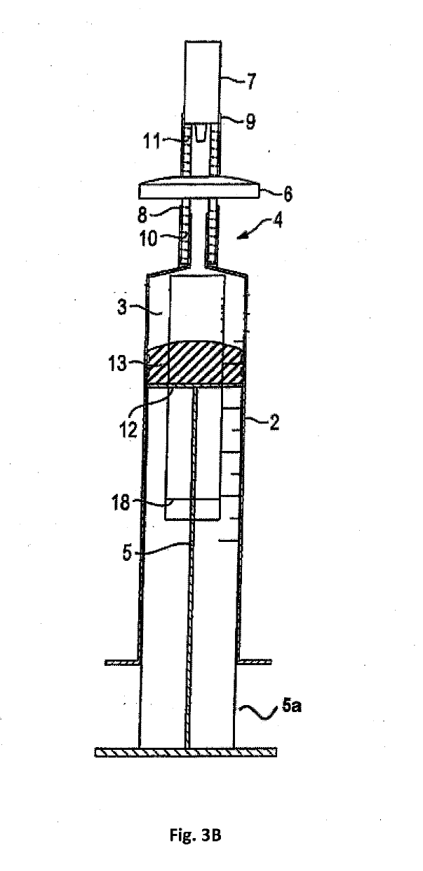

For all the embodiments with a (fillable) inner chamber volume of 100 ml, the predetermined amount of e.g. SF₆ is 20 ml, the amount of C₂F₆ is 16 ml and the amount of C₃F₈ is 12 ml. For smaller syringe volumes, e.g. 50 ml filling volume of the inner chamber 3, the amounts used are to be adapted proportionately. However, it is also possible to introduce smaller amounts of heavy gas. It is possible, for example, to use only 16 ml of SF₆ (based on a syringe with a filling volume of the inner chamber 3 of 100 ml) and 84 ml of air, i.e. a mixing ratio of 16% heavy gas/84% air for subsequent surgical use, e.g. in a vitrectomy. The present example includes preferred embodiments in which 16 ml of SF₆ and 84 ml of air are present as the gaseous mixture in a syringe according to the invention and can be administered immediately, as well as alternative embodiments in which only 16 ml of SF₆ are present in the syringe according to the invention and 84 ml of air are aspirated by directly pulling the plunger rod back. In this context, attention is drawn once again to the difference from the syringe known from European patent specification EP 2 319 566 B1. Thus, when using SF₆ as the heavy gas, the syringe known from EP 2 319 566 B1 with a filling volume of the inner chamber 3 of 100 ml is filled with 30 ml of SF₆ and delivered filled with this amount, so, after the excess heavy gas has been expelled and air aspirated (as shown in FIGS. 2A and 2B), 20 ml of SF₆ remain in the inner chamber 3 of the syringe and a mixing ratio of 20% gas/80% air is established. By contrast, the syringe according to the invention is preferably filled at the outset with the desired 20 ml of SF₆ and 80 ml of air; alternatively, it is filled with 20 ml of SF₆, and 80 ml of air are aspirated by pulling the plunger rod back. In general terms, in the present invention, the syringe is preferably filled at the outset with the amount of heavy gas and air, or alternatively heavy gas, which is required for the desired mixing ratio of heavy gas with air. This amount is also referred to here as the “predetermined amount” of heavy gas.

“Ready-to-use” implies that the syringe according to the invention is a ready-to-use system that does not require additional assembling and/or filling from an external gas container. The syringe according to the invention can thus be used directly in the manner described herein. This comprises, e.g. when using the syringe according to the invention in medical procedures, the attachment of a cannula or a tube immediately prior to injection of the heavy gas or heavy gas/air mixture.

The syringe according to the invention is preferably packed in a bag that is preferably gas-tight.

In conformance with the above disclosure, methods of filling a syringe with a predetermined amount of a heavy gas or a predetermined amount of a heavy gas and air (heavy gas/air mixture in a predetermined mixing ratio) are also disclosed here. Preferably, the syringe in this method is a syringe (1) in the form of a ready-to-use system with
a syringe body (2) having
   an inner chamber (3) and
   a tip (4) connected to the inner chamber (3),
a plunger (5a), guided in a longitudinally displaceable manner in the inner chamber (3), preferably with a plunger rod (5), and
a sealing stopper (7),
the inner chamber (3) taking the form of a gas container and being fillable with air and a heavy gas in a predetermined gas mixing ratio,
a syringe attachment filter (6) being fixed to the tip (4), and
the sealing stopper (7) being fixed to the syringe attachment filter (6), which seals the inner chamber (3) gas-tight,
the method comprising:
positioning of the plunger rod (5) or plunger (5)a, respectively, in such a way that the inner chamber (3) can be filled with a predetermined mixing ratio of a mixture of air and a heavy gas, and
filling of the inner chamber with amounts of air and heavy gas which are proportioned to give the desired mixing ratio so that the mixture of air and heavy gas is capable of immediate administration.

In conformance with the above disclosure, the heavy gas used can be SF₆, C₃F₈ or C₂F₆. The predetermined amount of heavy gas in the inner chamber 3 can be proportioned in such a way as to give the following mixing ratio of the heavy gas to the air that is also introduced after the syringe has been filled:
for SF₆: 20% gas/80% air,
for C₂F₆: 16% gas/84% air, and
for C₃F₈: 12% gas/88% air.

In conformance with the above disclosure, in contrast to the known syringe from FIG. 1, the syringe body 2 of the syringe 1 according to the invention preferably has no mark 18 for the maximum plunger stroke and/or no mixing ratio marks 15, 16 or 17 for different heavy gases.

The method according to the invention can also comprise the step of introducing the filled syringe into a bag and sealing the bag (gas-tight). In some embodiments, the bag used for this purpose is a bag made of a plastic material that is acceptable for pharmaceutical packs. Such a bag can be a sterile bag. The resulting system made up of a bag and a syringe packed inside is then conventionally sterilized, e.g. by autoclaving, with UV, X rays or gamma radiation or with formaldehyde, ethylene oxide, ozone or hydrogen peroxide, and shipped in sterile form to the user.

The bag with the syringe inside can also be evacuated by withdrawing air from it. For this purpose the bag is conventionally designed so as to be suitable for evacuation. This can imply, inter alia, that the bag is made of a vacuum-tight material, e.g. polyamide, polyethylene or laminated film.

Alternatively, it is also possible to use a bag that is not suitable for evacuation. In this case the bag can first be sterilized together with the syringe inside, as described above. The sterilized bag together with the syringe inside can then be packed in a second bag that is suitable for evacuation (“vacuum bag”). The vacuum bag can then be evacuated.

The present invention further comprises a method in which the syringe is a syringe (1) in the form of a ready-to-use system
with a syringe body (2) having
   an inner chamber (3) and
   a tip 4 connected to the inner chamber (3),
with a plunger (5a), guided in a longitudinally displaceable manner in the inner chamber (3), preferably with a plunger rod (5),
with a sealing stopper (7), and
with a mark (18) affixed to the syringe body (2) for the maximum plunger stroke,
the inner chamber (3) taking the form of a gas container and being Tillable with a predetermined amount of a heavy gas,
a syringe attachment filter (6) being fixed to the tip (4), and
the sealing stopper (7) being fixed to the syringe attachment filter (6), which seals the inner chamber (3) gas-tight.

The method comprises the step of aspirating air into the inner chamber 3, containing a predetermined amount of a heavy gas, by directly pulling the plunger rod 5 or plunger 5a, respectively, back to the mark 18 for the maximum plunger stroke to give a desired mixing ratio of the heavy gas with the aspirated air without the plunger rod 5 or plunger 5a, respectively, having been moved, prior to the aspiration of air, to a position in the inner chamber 3 where an excess amount of the heavy gas is expelled from the inner chamber 3 in order to establish the desired mixing ratio between the heavy gas and the air.

The method disclosed here further comprises the step, prior to the aspiration of air, of filling the inner chamber 3 of the syringe body with a predetermined amount of the heavy gas. In conformance with the above disclosure, the heavy gas used can be SF₆, C₃F₈ or C₂F₆. In this embodiment, the predetermined amount of heavy gas in the inner chamber 3 can be proportioned in such a way as to give the following mixing ratio of the heavy gas to the aspirated air after aspiration of the air by pulling the plunger rod back:
for SF₆: 20% gas/80% air,
for C₂F₆: 16% gas/84% air and
for C₃F₈: 12% gas/88% air.

In all the methods described here, in preferred embodiments with a volume of the inner chamber 3 of 100 ml, the predetermined (introduced) amount of SF₆ is 20 ml, the amount of C₂F₆ is 16 ml and the amount of C₃F₈ is 12 ml.

All the methods described here are methods of preparing a mixture of a heavy gas and air in a desired mixing ratio and at the same time methods of administering this mixture for medical purposes, e.g. an eye operation.

Those skilled in the art are aware that the syringe according to the invention is preferably sterile, especially for medical purposes. Provision is therefore made for the syringe according to the invention to be able to be prepared and filled under sterile conditions, and/or to be sterilized after preparation and filling, as described above.

Using the syringe according to the invention of one of the embodiments described above, a heavy gas/air mixture is administered to a patient's vitreous humour and/or vitreous body in order to effect a so-called gas tamponade (filling of the vitreous chamber). The gas tamponade is intended to press the retina on to the substrate. The retina is thus stabilized by the gas tamponade and smoothed out as required, and/or the closure of a macular foramen is supported.

The gas tamponade disappears from the eye over a period of approx. 1 week (air) to several weeks (heavy gas). The vitreous chamber is then refilled with collyrium, which is regularly produced by the ciliary body and replaces the vitreous humour.

The present invention therefore describes the use of a heavy gas for the preparation of a product, preferably a medical product such as, in particular, the syringe according to the invention. According to one embodiment of the invention, a predetermined amount of a heavy gas and air is filled into the product, as described herein, so that it can then be administered immediately by the user (e.g. eye surgeon) in this predetermined mixing ratio of heavy gas to air. According to an alternative embodiment, a predetermined amount of a heavy gas is filled into the product, as described herein, so that it can then be adjusted by the user (e.g. eye surgeon) to a specific heavy gas/air ratio.

The present invention further describes the use of a heavy gas for a device, e.g. a medical product, especially a syringe for injecting a surgical heavy gas into the vitreous humour and/or the vitreous body of an eye in order to prevent and/or treat retinal detachments, e.g. retinal detachments with giant tears, retinal detachments with proliferation, retinal detachments in cases of proliferative diabetic retinopathy (PDR), and traumatic retinal detachments. In other words, when used, the device is preferably filled with the heavy gas and air, or alternatively just with a heavy gas, as described herein, so that retinal detachments are prevented and/or treated with the ready-to-use system described herein.

In addition to the above written disclosure of the invention, explicit reference is hereby made to the drawings thereof and the explanations relating to the state of the art in FIGS. 1 to 3A, 3B.

### LIST OF REFERENCE NUMBERS

1 syringe

2 syringe body

3 inner chamber

4 tip

5 plunger rod

5a plunger

6 syringe attachment filter

7 sealing stopper

8 fixing section

9 fixing section

10, 11 screw connectors

13 rubber stopper

14 label

15, 16, 17 mixing ratio marks

18 mark for maximum plunger stroke

19 mixture of air and heavy gas

20 bag

1. A syringe (1) in the form of a ready-to-use system comprising
a syringe body (2) having
an inner chamber (3) and
a tip (4) connected to the inner chamber (3),
with a plunger (5a), guided in a longitudinally displaceable manner in the inner chamber (3), preferably with a plunger rod (5), and
with a sealing stopper (7),
the inner chamber (3) taking the form of a gas container and being filled with a mixture of air and a heavy gas in a predetermined mixing ratio, the plunger rod (5) or plunger (5a), respectively, being positioned in such a way that the mixture of air and heavy gas present in the predetermined mixing ratio is capable of immediate administration,
a syringe attachment filter (6) being fixed to the tip (4), and
the sealing stopper (7) being fixed to the syringe attachment filter (6), which seals the inner chamber (3) gas-tight,
the syringe body (2) not carrying a mark (18) for the maximum plunger stroke and/or one or more mixing ratio marks (15, 16 or 17) for different heavy gases.

2. The syringe according to claim 1 wherein the syringe attachment filter (6) has a first fixing section (8) for attachment to the tip (4) and a second fixing section (9) for securing the sealing stopper (7).

3. The syringe according to claim 1 wherein the syringe attachment filter (6) and the sealing stopper (7) are fixed by means of screw connectors (10 and 11 respectively).

4. The syringe according to claim 3 wherein the screw connectors (10 and 11) take the form of Luer lock connectors.

5. The syringe according to claim 1 wherein, at its end region (12) located in the inner chamber (3), the plunger rod (5) is provided with a rubber stopper (13).

6. The syringe according to claim 5 wherein the rubber stopper (13) is siliconized.

7. The syringe according to claim 1 which takes the form of a disposable syringe.

8. The syringe according to claim 1 wherein the heavy gas is SF₆, C₃F₈ or C₂F₆.

9. The syringe according to claim 8 wherein the heavy gas and air are present in the inner chamber (3) in the following mixing ratio:
for SF₆: 20% gas/80% air,
for C₂F₆: 16% gas/84% air, and
for C₃F₈: 12% gas/88% air.

10. The syringe according to claim 9 wherein, with a volume of the inner chamber (3) of 100 ml, the amount of SF₆ is 20 ml, the amount of C₂F₆ is 16 ml and the amount of C₃F₈ is 12 ml, the remainder being air.

11. The syringe according to claim 1 wherein the syringe attachment filter (6) is omitted and the sealing stopper (7) is fixed to the syringe in such a way that it seals the inner chamber (3) gas-tight.

12. A bag (20) with a syringe (1) packed inside, the latter being a syringe in the form of a ready-to-use system, comprising
a syringe body (2) having
an inner chamber (3) and
a tip (4) connected to the inner chamber (3),
with a plunger (5a), guided in a longitudinally displaceable manner in the inner chamber (3), preferably with a plunger rod (5), and
with a sealing stopper (7),
a syringe attachment filter (6) being fixed to the tip (4),
the sealing stopper (7) being fixed to the syringe attachment filter (6), which seals the inner chamber (3) gas-tight, and
the inner chamber (3) taking the form of a gas container and being filled with a mixture of air and a heavy gas in a predetermined mixing ratio, the plunger rod (5) or plunger (5a), respectively, being positioned in such a way that the mixture of air and heavy gas present in the predetermined mixing ratio is capable of immediate administration.

13. The bag according to claim 12 wherein the syringe attachment filter (6) is omitted and the sealing stopper (7) is fixed to the syringe in such a way that it seals the inner chamber (3) gas-tight.

14. A method of filling a syringe (1) with a predetermined amount of a heavy gas in the form of a ready-to-use system with
a syringe body (2) having
an inner chamber (3) and
a tip 4 connected to the inner chamber (3),
a plunger (5a), guided in a longitudinally displaceable manner in the inner chamber (3), preferably with a plunger rod (5), and
a sealing stopper (7),
the inner chamber (3) taking the form of a gas container and being fillable with air and a heavy gas in a predetermined gas mixing ratio,
a syringe attachment filter (6) being fixed to the tip (4), and
the sealing stopper (7) being fixed to the syringe attachment filter (6), which seals the inner chamber (3) gas-tight,
comprising the steps of:
positioning of the plunger rod (5) or plunger (5)a, respectively, in such a way that the inner chamber (3) can be filled with a predetermined mixing ratio of a mixture of air and a heavy gas, and
filling of the inner chamber with amounts of air and heavy gas which are proportioned to give the desired mixing ratio so that the mixture of air and heavy gas is capable of immediate administration.

15. The method according to claim 14 wherein the syringe attachment filter (6) is omitted and the sealing stopper (7) is fixed to the syringe in such a way that it seals the inner chamber (3) gas-tight.

16. A syringe according to claim 1 for use in a method of administering a heavy gas/air mixture into the vitreous humour.

17. A syringe according to claim 1 for use in the treatment or prevention of retinal detachments.
